(19) United States
(12) Patent Application Publication
(10) Pub. No.: US 2004/0116451 A1
(43) Pub. Date: Jun. 17, 2004

(54) Method of treatment

(75) Inventor: Ian William Mills, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/667,119

(22) Filed: Sep. 17, 2003

Related U.S. Application Data
(60) Provisional application No. 60/417,520, filed on Oct. 9, 2002.

(30) Foreign Application Priority Data
Sep. 17, 2002 (GB) 0221582.0

(51) Int. Cl. A61K 31/517

(52) U.S. Cl. 514/266.22

(57) ABSTRACT

The use of 4-amino-6,7-dimethoxy-2-(5-methanesulfonamido-1,2,3,4-tetrahydroisoquinol-2-yl)-5-(2-pyridyl)quinazoline, or a pharmaceutically acceptable derivative thereof, for treating LUTS associated with: OAB (with or without concomitant detrusor over activity); pelvic floor dysfunction; or chronic prostatitis, is described.

This application is filed claiming priority to U.S. Provisional Serial No. 60/417,520, filed Oct. 9, 2002, and GB Application Serial No. 0221582.0, filed Sep. 17, 2002.

This invention relates to a new use of 4-amino-6,7-dimethoxy-2-(5-methanesulfonamido-1,2,3,4-tetrahydroisoquinol-2-yl)-5-(2-pyridyl)quinazoline (disclosed as example 19 in International Patent Application Publication No. WO 98/30560), and its pharmaceutically acceptable derivatives. The mesylate salt is disclosed in International Patent Application Publication No. WO 01/64672 (e.g. Example 2). Both WO 98/30560 and WO 01/64672 are incorporated herein by reference. It is indicated in the treatment of Benign Prostatic Hyperplasia (BPH) and has the following structure:

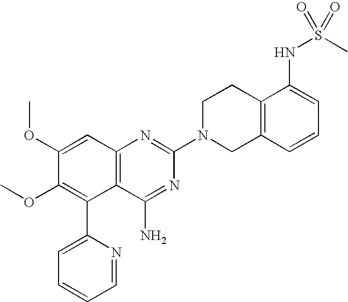

Lower urinary tract symptoms (LUTS) comprise three groups of symptoms, which are irritative, obstructive and post micturition symptoms. Irritative symptoms comprise urgency, frequency and nocturia, which can be associated with: overactive bladder (with or without concomitant detrusor over activity); pelvic floor dysfunction; or chronic prostatitis.

Over Active Bladder (OAB) is defined as urgency, with or without urge incontinence, usually with frequency and nocturia [Abrams et al., Neurourology and Urodynamics 21:167-178 (2002)]. Prevalence of OAB in men and women is similar, with approximately 16% of the population of the USA suffering from the condition [Stewart et al, Prevalence of Overactive Bladder in the United States: Results from the NOBLE Program; Abstract Presented at the 2^{nd} International Consultation on Incontinence, July 2001, Paris, France].

Pelvic floor dysfunction (PFD) occurs when the muscles of the pelvic floor no longer relax properly during urination while the bladder contracts. The muscles may become irritated and often contract abnormally. PFD may result in irritative LUTS.

Chronic prostatitis is an inflammatory condition of the prostate, which may or may not be associated with uropathogenic bacteria detected by standard microbiological methodology. It is characterized by the presence of genitourinary pain or discomfort, often associated with irritative LUTS.

Overactive bladder may be suffered by individuals of any age, while pelvic floor dysfunction and prostatitis are conditions typically suffered by middle-aged men. Patients with any of these conditions are likely to experience irritative lower urinary tract symptoms, and often the eventual diagnosis is empirical.

Surprisingly it has been found that 4-amino-6,7-dimethoxy-2-(5-methanesulfonamido-1,2,3,4-tetrahydroisoquinol-2-yl)-5-(2-pyridyl)quinazoline is useful in the treatment of LUTS associated with: OAB (with or without concomitant detrusor over activity); pelvic floor dysfunction; or chronic prostatitis.

Thus, in accordance with the present invention, there is provided the use of 4-amino-6,7-dimethoxy-2-(5-methanesulfonamido-1,2,3,4-tetrahydroisoquinol-2-yl)-5-(2-pyridyl)quinazoline, or a pharmaceutically acceptable derivative thereof, for the manufacture of a medicament for the treatment of LUTS associated with: OAB (with or without concomitant detrusor over activity); pelvic floor dysfunction; or chronic prostatitis.

Preferably the LUTS is associated with pelvic floor dysfunction. Alternatively, the LUTS is preferably associated with chronic prostatitis.

Preferably the 4-amino-6,7-dimethoxy-2-(5-methanesulfonamido-1,2,3,4-tetrahydroisoquinol-2-yl)-5-(2-pyridyl)quinazoline is in the form of its mesylate salt.

The compound, or a pharmaceutically acceptable derivative thereof, can be administered alone or in any convenient pharmaceutical presentation. Oral administration is preferred. In the present indication, a suitable dosage of the compound, or of the active moiety in a pharmaceutically acceptable derivative thereof, is from about 0.01 to 10.0 mg/kg of body weight, and preferably about 0.05 to 1.0 mg/kg is suitable. Administration may be in single does of from 1 to 4 times daily or preferably it may be in a controlled release formulation such as is disclosed in International Application Publication No. WO 03/032956 (see in particular examples 1 to 5). Administration may be p.r.n. for occasions when the patient may have limited access to toilet facilities, e.g. during a long journey.

The invention further provides 4-amino-6,7-dimethoxy-2-(5-methanesulfonamido-1,2,3,4-tetrahydroisoquinol-2-yl)-5-(2-pyridyl)quinazoline, or a pharmaceutically acceptable derivative thereof, for use in the treatment of LUTS associated with: OAB (with or without concomitant detrusor over activity); pelvic floor dysfunction; or chronic prostatitis.

The invention further provides a method of treating LUTS associated with: OAB (with or without concomitant detrusor over activity); pelvic floor dysfunction; or chronic prostatitis, which comprises administering 4-amino-6,7-dimethoxy-2-(5-methanesulfonamido-1,2,3,4-tetrahydroisoquinol-2-yl)-5-(2-pyridy!)quinazoline, or a pharmaceutically acceptable derivative thereof, to a patient in need of such treatment.

### EXAMPLES 1-5

### Tablet Formulations of 4-Amino-6,7-dimethoxy-2-(5-methanesulfonamido-1,2,3,4-tetrahydroisoquinol-2-yl)-5-(2-pyridyl)quinazoline mesylate containing Methocel™ K4M

The following table shows the ingredients for preparing five tablet formulations containing, respectively, 1, 3, 6, 9 and 12 mg of active ingredient, expressed as free base, according to International Application Publication No. WO 03/032956.

| Ingredient (mg) | | | | | |
|---|---|---|---|---|---|
| (reference to standard) | Ex 1 | Ex 2 | Ex 3 | Ex 4 | Ex 5 |
| 4-amino-6,7-dimethoxy-2- | 1.189⁽¹⁾ | 3.567 | 7.134 | 10.701 | 14.268 |
| (5-methanesulfonamido- | | | | | |
| 1,2,3,4-tetrahydroisoquinol- | | | | | |
| 2-yl)-5-(2-pyridyl)quinazoline | | | | | |
| mesylate (Pfizer) | | | | | |
| HPMC | 30.000 | 30.000 | 30.000 | 22.500 | 22.500 |
| (Methocel K4M, Ph. Eur) | | | | | |
| Lactose Monohydrate | 13.203 | 10.108 | 9.216 | 10.200 | 9.308 |
| (Ph. Eur) | | | | | |
| Calcium Hydrogen | 39.608 | 30.325 | 27.650 | 30.599 | 27.924 |
| Phosphate, Anhydrous | | | | | |
| (Ph. Eur) | | | | | |
| Adipic Acid | 15.000 | 25.000 | 25.000 | 25.000 | 25.000 |
| [DAB⁽²⁾] | | | | | |
| Magnesium Stearate | 1.000 | 1.000 | 1.000 | 1.000 | 1.000 |
| (Ph. Eur) | | | | | |
| Tablet weight (mg) | 100.000 | 100.000 | 100.000 | 100.000 | 100.000 |

⁽¹⁾Equivalent to 1.0 mg 4-amino-6,7-dimethoxy-2-(5-methanesulfonamido-1,2,3,4-tetrahydroisoquinol-2-yl)-5-(2-pyridyl)quinazoline, in the form of its free base
⁽²⁾DAB is the Deutsches Arzneibuch (German Pharmacopoeia)

The adipic acid was first screened through a suitable screen (e.g. 500 micron). The lactose monohyd rate, hydroxypropylmethyl cellulose, 4-amino-6,7-dimethoxy-2-(5-methanesulfonamido-1,2,3,4-tetrahydroisoquinol-2-yl)-5-(2-pyridyl)quinazoline mesylate, the screened adipic acid and calcium hydrogen phosphate, anhydrous were then added to a suitable blender (e.g. a tumble mixer) and blended. The blend was screened through a suitable screen (e.g. 500 micron) and reblended. About 50% of the lubricant (magnesium stearate) was screened, added to the blend and blended briefly.

The blend was roller compacted through a suitable roller compactor. The ribbon blend was then granulated, by screening through a suitable screen (e.g. 500 micron) and reblended. The remaining lubricant was screened, added to the blend and blended briefly.

The granules were then tabletted using appropriate 6 mm tooling to give 6 mm standard round convex white tablets with no engraving, which were then de-dusted.

### EXAMPLE 6

### In Vivo Study

A 12-Week Study in men with lower urinary tract symptoms was undertaken in which the IPSS (International Prostate Symptom Score) was recorded at baseline during, and at the end of, double-blind treatment. The IPSS is composed of seven questions, each with potential responses of 0-5 on a Likert scale. These questions are grouped into two validated domains: the irritative domain (urgency, frequency and nocturia) and the obstructive domain (incomplete emptying, intermittency, weak stream and straining to begin). In addition, a bladder diary was completed by each subject to provide baseline incidence of individual symptoms, and subsequently to demonstrate change in incidence of these symptoms following double blind treatment. The average daily incidence of urgency, daytime micturition frequency and nocturia (the irritative symptoms) for each subject were derived from this diary. In this study, there were five treatment groups: 6 mg fixed dose of 4-amino-6,7-dimethoxy-2-(5-methanesulfonamido-1,2,3,4-tetrahydroisoquinol-2-yl)-5-(2-pyridyl)quinazoline, 6 mg escalated to 12 mg at Week 4 of the compound, and placebo. Controlled release formulations according to International Application Publication No. WO 03/032956 were used in each case.

For those subjects with irritative LUTS at baseline, improvement in these symptoms was confirmed in the compound 6 mg fixed dose group and the 12 mg dose escalation group, compared with the placebo treated group. In subjects with baseline IPSS irritative domain score ≧β at baseline, improvement in this domain of the IPSS was similarly confirmed in both the compound 6 mg fixed dose group and the 12 mg dose escalation group.

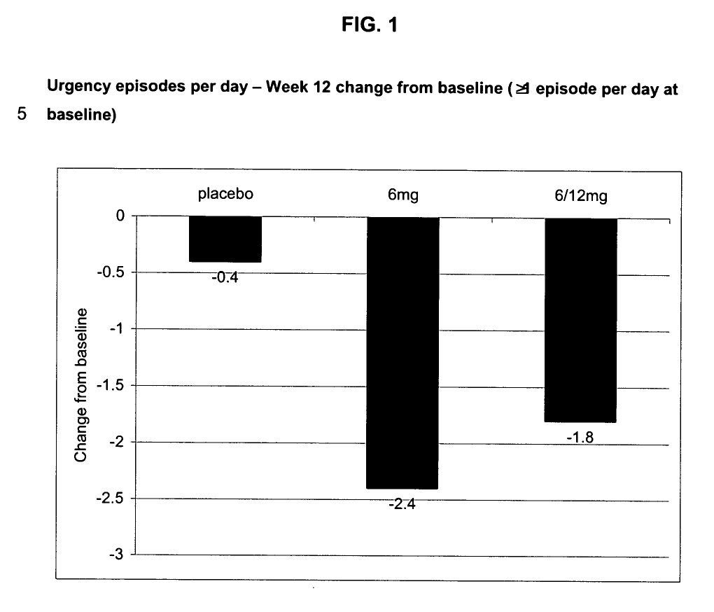

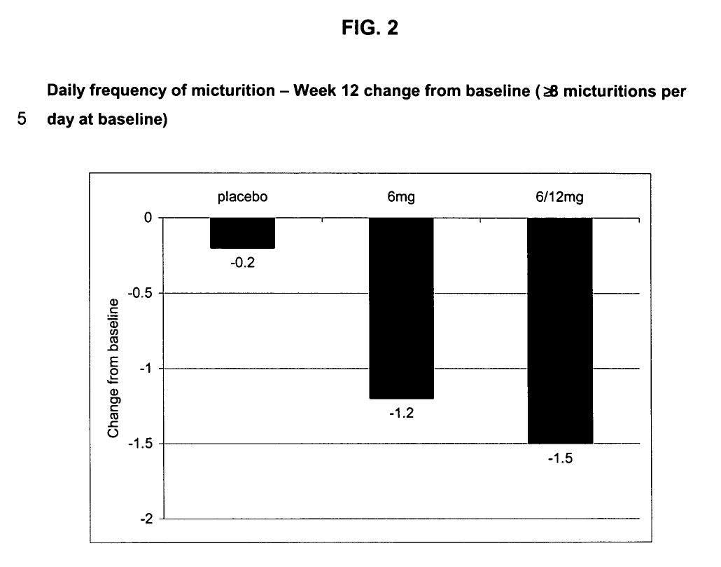

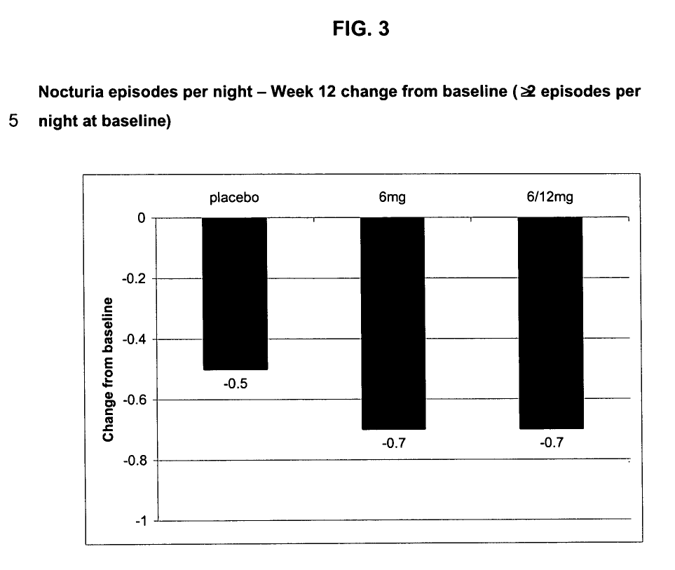

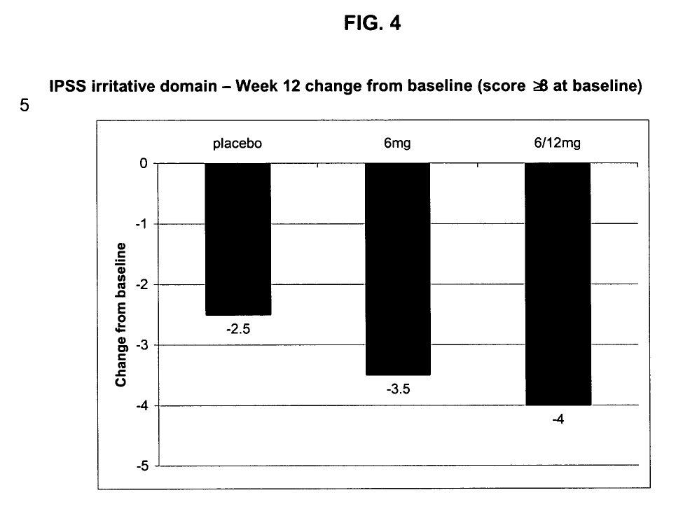

The results of the study are illustrated in FIGS. 1 to 4 which show 4-amino-6,7-dimethoxy-2-(5-methanesulfonamido-1,2,3,4-tetrahydroisoquinol-2-yl)-5-(2-pyridyl)quinazoline produced a clinically significant attenuation of irritative LUTS.

1. A method of treating LUTS associated with: OAB, with or without concomitant detrusor over activity; pelvic floor dysfunction; or chronic prostatitis, comprising administering 4-amino-6,7-dimethoxy-2-(5-methanesulfonamido-1,2,3,4-tetrahydroisoquinol-2-yl)-5-(2-pyridyl)quinazoline, or a pharmaceutically acceptable derivative thereof, to a patient in need of such treatment.

2. A method as claimed in claim 1, wherein the 4-amino-6,7-dimethoxy-2-(5-methanesulfonamido-1,2,3,4-tetrahydroisoquinol-2-yl)-5-(2-pyridyl)quinazoline is in the form of its mesylate salt.

3. A method as claimed in claim 1, wherein the LUTS is associated with pelvic floor dysfunction.

4. A method as claimed in claim 1, wherein the LUTS is associated with chronic prostatitis.
